(19) United States
(12) Patent Application Publication
(10) Pub. No.: US 2010/0106228 A1
(43) Pub. Date: Apr. 29, 2010

(54) DEVICE AND METHOD OF PHOTOTHERAPY FOR JAUNDICED INFANTS

(76) Inventor: Steven Gardner, Chapin, SC (US)

Correspondence Address: BROOKS KUSHMAN P.C., 1000 TOWN CENTER, TWENTY-SECOND FLOOR, SOUTHFIELD, MI, 48075, US

(21) Appl. No.: 12/258,857

(22) Filed: Oct. 27, 2008

(51) Int. Cl. A61N 5/06

(52) U.S. Cl. 607/90

(57) ABSTRACT

A phototherapy device and method for treating neonatal hyperbilirubinemia (jaundice) and related conditions. The device comprises a flexible material at least partially encasing flexible circuitry that allows the device to flex as an infant is positioned on it, while, also protecting the circuitry. The circuitry comprises a plurality of light emitting diodes (LEDs) mounted within flexible circuitry, means for altering the duty cycle of the LEDs, and wiring to connect the circuitry to a power supply. The device further comprises a multilayer film that encases all of the circuitry, the flexible material, and LEDs to facilitate sterilization in both long-term or short-term use.

### BACKGROUND OF THE INVENTION

1. Field of the Invention

This invention relates generally to the treatment of neonatal hyperbilirubinemia (jaundice), and more specifically it relates to phototherapy treatment methods and devices.

2. Background Art

Approximately 60% of infants born in the United States each year become clinically jaundiced. Jaundice, or hyperbilirubinemia, results from increased production and transiently impaired elimination of bilirubin. While most affected neonates recover rapidly, some infants show persistently high levels of unconjugated bilirubin. Such high levels can lead to kernicterus, a condition involving deposition of bilirubin in the brain, which leads to deficits in cognition, neuromuscular tone and control, hearing, and even death. The most common therapy for neonatal hyperbilirubinemia is phototherapy. It is estimated that as many as 400,000 neonates in the United States receive phototherapy every year. Phototherapy facilitates the transformation of unconjugated bilirubin to compounds that are more easily excreted.

Phototherapy for treating hyperbilirubinemia is commonly delivered using fluorescent lamps suspended above the neonate. However, fluorescent lamps generate heat (infrared radiation), which prevents their placement close to the infant, thereby decreasing the irradiance. Fluorescent light is of a broad spectral range, and cannot be produced in the narrow wavelength range desired. Conventional phototherapy devices typically illuminate the newborn only from above, and do not therefore make optimal use of the available skin area.

The use of fluorescent lamps for phototherapy leads to adverse side effects in many newborns. Such side effects include increased insensible water loss, hypothermia, loose and frequent bowel movements, tanning, and potential nasal obstruction by the eye pads required for preventing retinal damage. Furthermore, there are concerns that phototherapy using fluorescent lamps has potentially harmful effects on biological rhythms, and may increase the incidence of skin cancer in neonates subject to repeated treatment.

### SUMMARY OF THE INVENTION

The present invention provides a phototherapy device and method for treating neonatal hyperbilirubinemia (jaundice) and related conditions, such as Crigler-Najjar Syndrome. The present invention is an improved phototherapy device and method for treating neonatal hyperbilirubinemia. The device comprises a flexible material that at least partially encases flexible circuitry which allows an infant to be held and carried by a caregiver while the infant is undergoing treatment. The circuitry comprises a plurality of light emitting diodes (LEDs) mounted in a pattern, flexible circuitry, means to alter the duty cycle of the LEDs, and wiring to connect the circuitry to a power supply. The device further comprises a film encasing all of the circuitry, the flexible material, and LEDs that facilitates sterilization.

### BRIEF DESCRIPTION OF THE DRAWINGS

FIG. 1 is a perspective view of the phototherapy panel showing the general features;

FIG. 2 is a top view of the LED light panel;

FIG. 3 is a side view of the LED light panel;

FIG. 4 is a side view of an additional embodiment of the LED light panel;

FIG. 5 is a top view of the device having an outer casing made from a multilayer film in the form of a bag;

FIG. 6 is a top view of the device having an outer casing made from a multilayer film in the form of an IV bag;

FIG. 7 is a top view of the device having an outer casing made from a multilayer film in the form of a tube;

FIG. 8 is a schematic cross-section of a possible five-layer film that can be used to encase the device components;

FIG. 9 is an alternative embodiment of a LED light panel detailing the potted circuitry;

FIG. 10 is a diagram of a process used to create a phototherapy panel consistent with an embodiment of the present invention; and

FIG. 11 is a diagram of another process used to create a phototherapy panel consistent with an embodiment of the present invention.

### DETAILED DESCRIPTION OF THE PREFERRED EMBODIMENT(S)

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for the claims and/or as a representative basis for teaching one skilled in the art to utilize the present invention.

Although the following detailed description contains many specifics for the purposes of illustration, and variations and alterations to the following details are within the scope of the invention. Accordingly, the following embodiments of the invention are set forth without any loss of generality to, and without imposing limitations upon, the claimed invention.

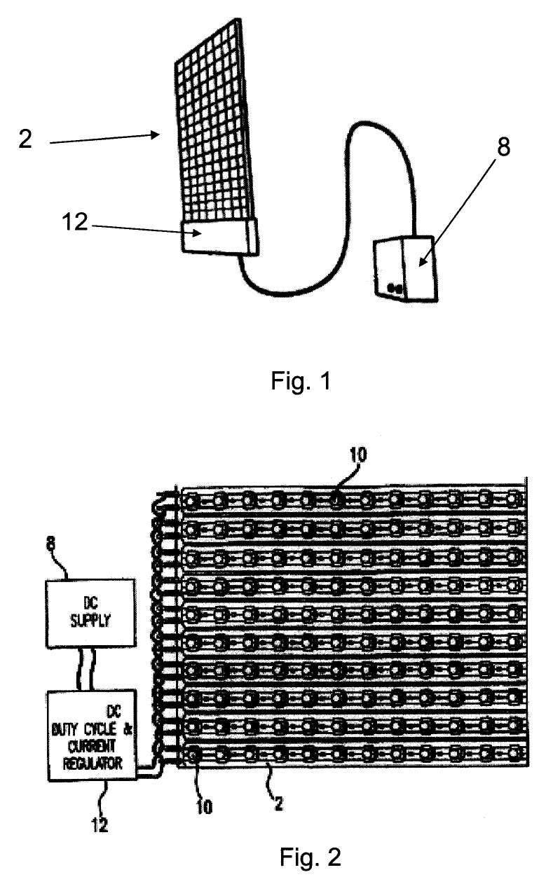

FIG. 1 shows the phototherapy panel 2 of the phototherapy device. The panel, which includes light emitting diodes (LEDs), a flexible circuitry substrate, and control circuitry 12, is preferred to be slightly larger than the length of the back of an infant. A power supply unit 8 powers the device.

In one embodiment, the phototherapy device comprises a flexible backing material 14, a transparent liner 18, and a flexible circuitry substrate 22, with light emitting diodes (LEDs) 10 mounted to the flexible circuitry substrate and conductively connected to a power supply 8. An infant is placed over the panel, with the LEDs emitting light toward the infant's back. The panel is adapted to provide light exposure by means of the LEDs over 100% of the infant's back, or somewhat beyond the infant's back. The phototherapy panel will effectively treat neonatal hyperbilirubinemia, also known as jaundice, via phototherapy.

It is preferred that the portion of the panel 2 in which LEDs are present is wider and longer than the infant's back. However, the panel is preferred to not be substantially longer or wider than is necessary to provide phototherapy to the infant's back. The portion of the panel comprising LED's (the treatment area) is preferred to not be larger than 12 cm by 18 cm. The thickness of the panel is preferred to be 1-2 cm.

The panel as constructed is preferred to be easily portable, that is, the infant may be carried with panel in place against the infant's back. The infant may be comforted and/or transported by a caregiver while the device is in use. Accordingly, the device is constructed so that it is as small as possible, while still providing efficacy. Further, the device as constructed in the preferred embodiment does not need cooling, such as by fans, fins, channels, or any other heat removal method or device, but it is preferred to keep the number of LEDs to a minimum number that is efficacious so that heat is not detrimental to the device or the infant.

In the preferred embodiment, the LEDs are preferred to emit high-intensity blue light suitable for treating neonatal hyperbilirubinemia. In another embodiment, the LEDs can be arranged in groups of different colors so that the color balance can be modified via the control circuitry varying the relative power to the LED groups to facilitate greater efficacy or the treatment of other medical conditions that can benefit from phototherapy. The LEDs are arranged to optimize intensity and coverage. The unit is preferred to have not fewer than one LED per 2.5 square centimeter within region of the matrix where LEDs are present.

A Power supply 8 is provided. The power supply can be an AC power supply and/or a DC power supply for higher degrees of portability. The portable power supply may be one or more batteries that supply direct current to the LEDs, and enable the unit to be highly portable. The portable power supply is low voltage direct current in the preferred embodiment, thereby reducing the risk of harmful electric shock to the patient. The AC power supply will use a standard transformer plug to reduce the power level and match that of the DC power supply and may be combined with a battery charger current and battery pouch pack to render the unit portable. Silicon chips enable the LEDs to have their duty cycle altered from 100% actuation time to as little as 10%, thereby increasing battery life, as well as decreasing the amount of heat generated.

The invention provides doctors and parents with a new device that combines the benefits of a fiber-optic panel and a phototherapy bed. The greatest benefit of the fiber-optic panel is that the parent is able to hold the infant without interrupting treatment, thereby supporting parent-infant bonding. The benefit of the device is that it covers more of the infants back due to its size. The invention is highly portable, has a dense coverage pattern, and intensity levels potentially exceeding 120 μW/cm²/nm (microwatts per centimeter squared per nanometer), which is nearly double the current output of a neonatal fiber optic panel or a phototherapy bed while supporting parent-infant bonding, as well as maintaining a high coverage area.

The use of light-emitting diodes (LEDs) is used in a preferred embodiment to deliver light directly to the neonate's skin. The LEDs are very small, very durable, and long lasting. As a result, the panel comprising the LEDs is portable, lightweight, comfortable, easy to use, and relatively inexpensive. LEDs deliver relatively high light intensity for their physical size and weight, with relatively low power consumption (e.g., 70 mW), and therefore have high efficiency (optical output power/electrical input power). They produce no harmful UV radiation and negligible heat (infrared). The LEDs may emit light of a wavelength suitable for treating hyperbilirubinemia in neonates, such as 420-500 nm, preferably between 440 and 480 nm. Each lens type LED emits light substantially all of its light throughout an included angle of about 90 to about 130 degrees and preferably throughout an angle of 90 to 110 degrees, however the viewable angle is not limited to these viewing angles. In one preferred embodiment, the invention will utilize LEDs that have a wavelength output between 450 and 470 nm, with a viewing angle of 130 degrees. It should be appreciated that minimal light is emitted at wider angles which is wasted energy adding to heat loss.

In a preferred embodiment, the present invention uses lens type LEDs. The LEDs that will be used are not limited to a specific type of LED. For example, low profile lens type LEDs or organic LEDs (OLED) can be utilized if the light output is sufficient to meet the output requirements for successful treatment. Additionally, the LEDs can be either surface mount or through hole mount. A phototherapy panel is made of a flexible material that incorporates the LEDs. The panel has a lining inside surface of reflective Mylar that reflects ambient light that is previously reflected from the neonate's skin.

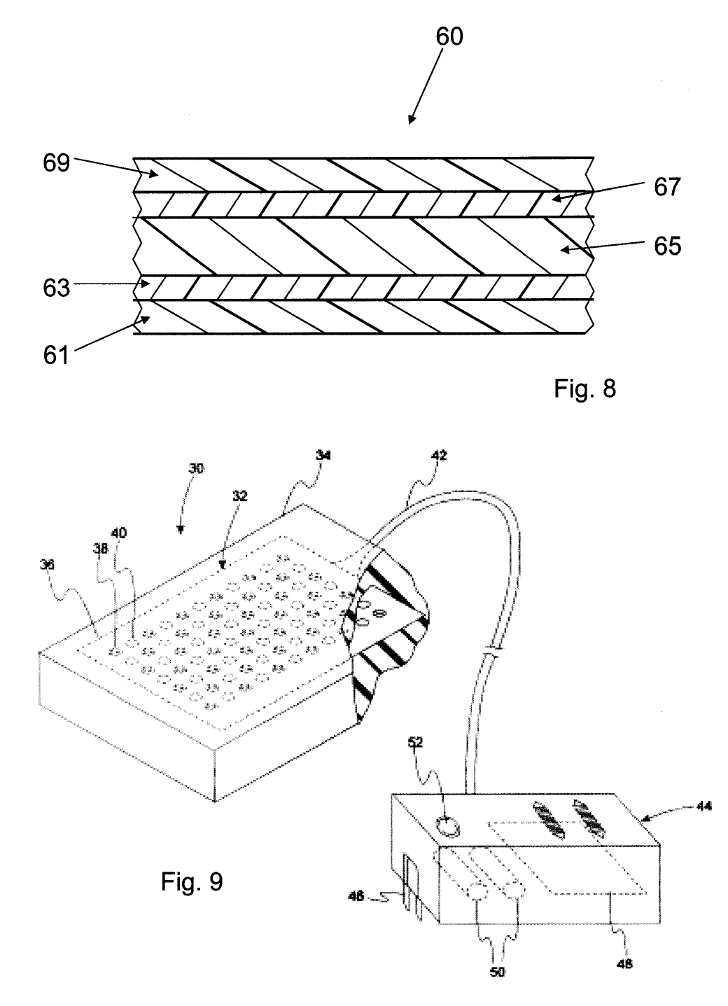

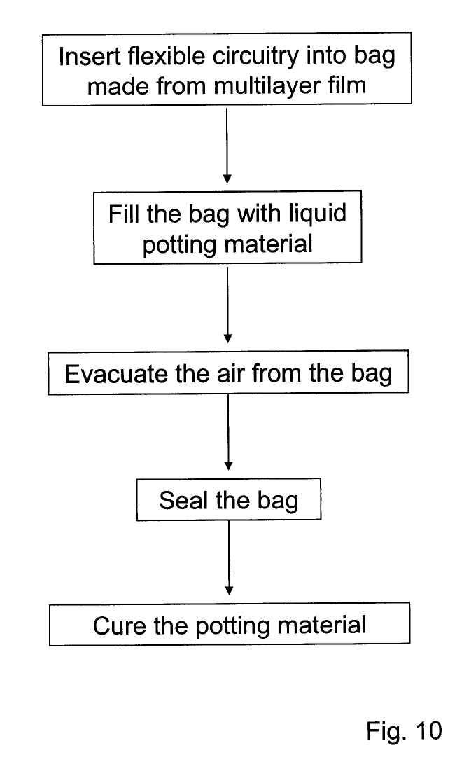

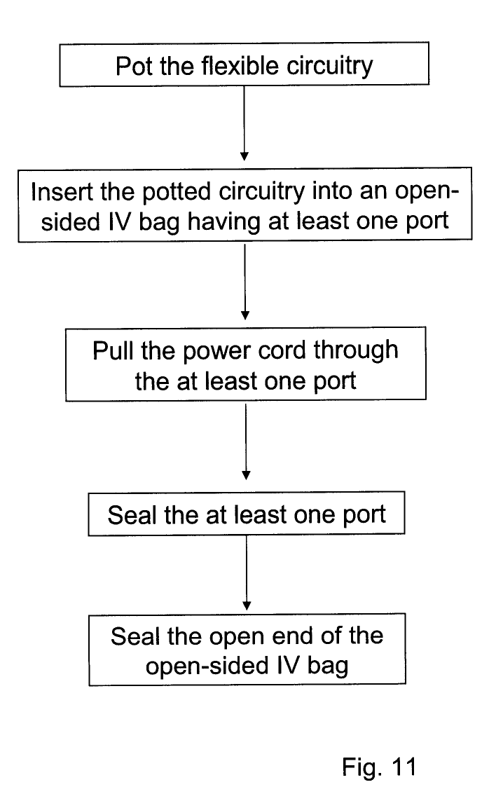

In one embodiment, the phototherapy panel is encased by a multilayer film 60 in the form of a bag 74 with one open end. The multilayer film is heat resistant and able to withstand heat sterilization at temperatures of at least about 121° C. as well as gamma radiation sterilization. The multilayer film preferably comprises at least one polyolefin layer. An example of a suitable multilayer film is the Cryovac® M312 Sterilizable Medical Film, which is currently used for IV bags. The composition of the Cryovac® M312 Sterilizable Medical Film is described in detail in U.S. Pat. No. 6,500,505 entitled “Thermoplastic Film with Good Interply Adhesion,” which is incorporated by reference herein. Alternative films are illustrated in U.S. Pat. Nos. 5,695,840 and 6,916,750. One of the outer layers of the film 61 includes polypropylene mer units. A second layer 63, directly adhered to the polypropylene outer layer, is a homogeneous ethylene/α-olefin interpolymer having a density of no more than about 0.915 g/cm³. Additional layers can be added to impart various properties, such as heat and/or abuse resistance, moisture barriers, and bulk layers. For example, FIG. 8 shows a five layer film with the outer layer 61, second layer 63, a moisture barrier layer 65, an adhesive layer 67, and a heat/abuse layer 69.

In another embodiment, the phototherapy panel 2 is inserted into a bag 74 formed from the multilayer film, with the power cord 42 extending beyond the cut end. A flexible material, such as silicone, may then be poured into the bag to encompass the phototherapy panel and at least part of the circuitry. The bag can then be subjected to a vacuum to remove the air from the bag and the bag can subsequently be heat sealed. A gap may exist around the power cord after the heat seal. To fill the space of the gap, more of the flexible material can be injected therein. To seal the gap, another material may be injected, such as a flexible epoxy. The external multilayer film will not hinder the flexibility of the panel, nor will the material hinder the light output of the needed wavelength for successful treatment.

In another embodiment, the multilayer film, such as the Cryovac® M312 Sterilizable Medical Film, is in the form of a pre-made IV bag 70, or similar type of bag, with one or more ports on one end. The non-ported end of the bag may be cut off to create an open bag. The phototherapy panel 2 may then be inserted into the bag, with the power cord 42 extending out from a port 71 in the IV bag. The port(s) can then be sealed with a flexible epoxy, or any other suitable sealant, and the flexible material can be poured into the bag to encompass the LEDs and at least a part of the flexible circuitry substrate and control circuitry. Vacuum may then be applied to remove the air from the bag and the open end can be heat sealed.

In another embodiment, the bags, either IV or open, may be filled and sealed as described above but without the step of the application of vacuum. Excess air may be removed as much as possible by other means, such as agitation, compression, or any other suitable method. There will likely still be some air left in the bag, but this may be acceptable, especially if the phototherapy device is to be disposable or for short-term use. Removing the application of vacuum step would likely decrease the time and cost necessary to produce the phototherapy device.

In another embodiment, the same multilayer film, such as the Cryovac® M312 Sterilizable Medical Film, is used to encase the phototherapy panel by heat shrinking the film around the panel. The phototherapy panel may be partially or fully potted in the flexible material, such as silicone, and then inserted into a tube 72 of the multilayer film, with the power cord extending beyond the end of the tube. The multilayer film may then be heat shrunk until it is tightly encases the phototherapy panel. If a gap exists in the seal around the power cord then a sealant may be introduced to fill the gap. The multilayer film can optionally be removed and replaced to maintain sterile conditions.

In another embodiment, a phototherapy panel may be partially or fully potted in a flexible material, such as silicone, and then inserted into an IV bag with a side removed made of the multilayer film, with the power cord extending through the port of the IV bag. The port of the IV bag can then be sealed. The air in the bag can then optionally be evacuated, either by vacuum or any other suitable method, and the open side can be sealed. In an alternative embodiment, additional potting material can be introduced into the IV bag before it is sealed.

In another embodiment, a phototherapy panel may be partially or fully potted in a flexible material, such as silicone, and then heat shrunk in a polymer film. The heat shrunk phototherapy panel may then be inserted into an IV bag with a side removed made of a multilayer film, with the power cord extending through the port of the IV bag. The port of the IV bag can then be sealed. The air in the bag can then optionally be evacuated, either by vacuum or any other suitable method, and the open side can be sealed.

In another embodiment, a phototherapy panel may be partially or fully potted in the flexible material, such as silicone, and then inserted into a bag made of the multilayer film, with the power cord extending out the open side of the bag. The air in the bag can then be evacuated, either by vacuum or any other suitable method, and the open side can be sealed. If a gap exists around the power cord then it can be sealed with any suitable sealant.

The flexible material 14, such as silicone gel, fully encapsulates the tops of the LEDs and the reflective sheet and at least part of the flexible printed circuit and control circuits, thereby creating a layer of flexible gel that encompasses the light emitting portions of the device completely. In the preferred embodiment, the power connection will be resistant to fluids, thus eliminating any threat of electrical shock for the patient when in use. The gel is preferred to be transparent or substantially transparent to the selected efficacious wavelength of the LEDs. Additionally, the exterior multilayer film may be coated with an anti-bacterial coating, such as MEDIGARD, manufactured by the Hydrogiene Corporation of San Diego, Calif. Medigard is formulated to kill a wide spectrum of bacteria, is resistant to cleaning and sterilization processes, and has a reputed four-year killing period. It can be coated in such a way as to be impossible to detach or destroy when applied to materials.

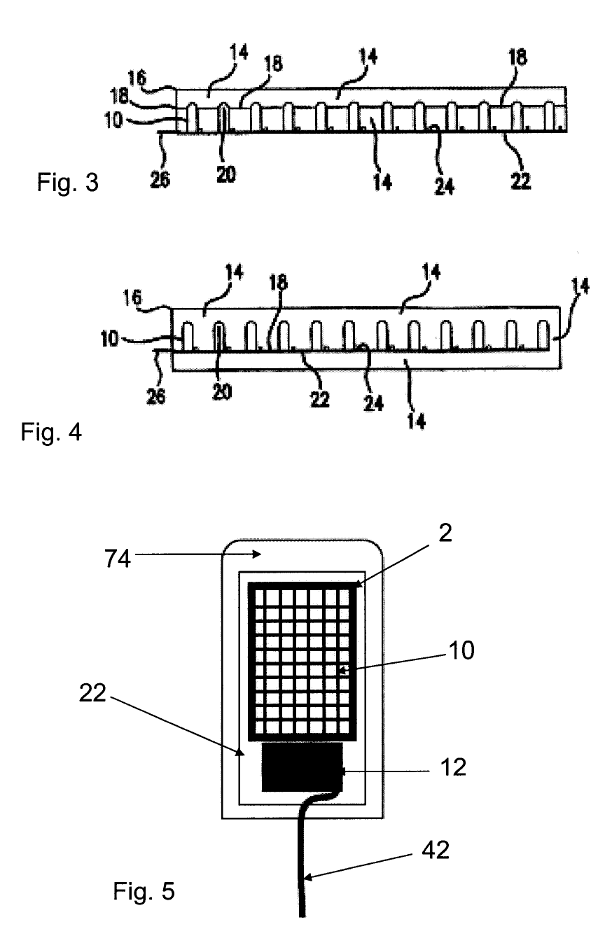

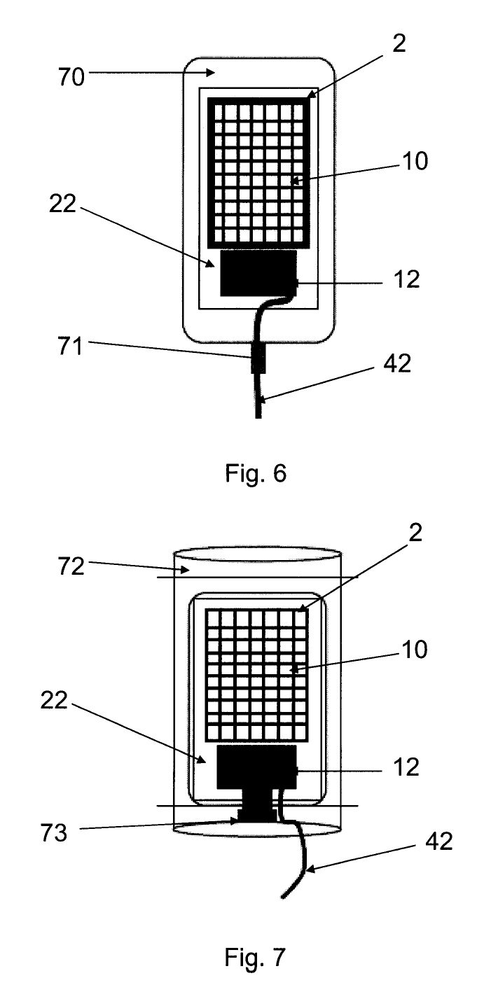

As shown in FIG. 3, the tops of the plurality of the LEDs are fully encapsulated by the substantially transparent gel 14. A reflective sheet 18 surrounds the plurality of LEDs 10. The reflective sheet is below the tops 20 of the LEDs so that light from the LEDs is reflected toward the top planar surface 16 of the panel. The reflective sheet may have a plurality of holes formed therein that are spaced to accept the LEDs within the holes. The reflective sheet will have enough space around the perforations to allow both the light of the LEDs to pass through as well as to allow the flexible gel to pass through thus forming a continuous single layer of flexible encapsulating gel. The reflective sheet is preferred to be not be materially electrically conductive, and may be MYLAR having a silver or white colored top surface. In another embodiment, the flexible substrate's 22 top surface would be white in color thereby eliminating the need for a MYLAR reflective sheet. The flexible circuitry substrate 22 is flexible, and has conductors 24 printed thereon that provide power to the LEDs. The gel also surrounds the LEDs below the reflective sheet and above the flexible circuitry substrate, so that the LEDs are encapsulated, with no spaces or pockets having a material size around or above the LEDs. Additionally, the flexible substrate 22 can have holes strategically placed to allow more of the flexible encapsulating gel to pass through flexible substrate 22 to promote greater structural integrity.

As shown in FIG. 4, which is an additional embodiment from the embodiment of FIG. 3, the plurality of the LEDs 10 are fully encapsulated by the substantially transparent gel 14. A reflective sheet 18 surrounds the plurality of LEDs. The reflective sheet is adjacent to and above the flexible circuitry substrate, so that light from the LEDs is reflected toward the top planar surface 16 of the panel. The reflective sheet is preferred to not be materially electrically conductive, and may be MYLAR having a silver or white colored top surface. The non-conductive reflective sheet may be in contact with the flexible circuitry substrate. The reflective sheet may have a plurality of holes formed therein that are spaced to accept the LEDs within the holes. The flexible circuitry substrate is flexible, and has conductors printed thereon that provide power to the LEDs. The gel is also present below the reflective sheet and the flexible circuitry substrate. At least a portion of the circuitry and all of the LEDs are encapsulated/potted by the gel, while the power supply and leads 26 to the power supply are preferably not encapsulated to reduce heat. No spaces or pockets of material size are around or above the LEDs, other than perhaps air bubbles from the formation process. Lead 26 extends from the gel material and communicates with the LEDs by a printed circuit on the flexible circuitry substrate.

In the preferred embodiment, the device can use either surface mount or through-hole mount LEDs. Each panel is preferred to contain not more than 70 low profile lens type LEDs that do not exceed 2.5 mm in height. The LEDs are mounted on a flexible circuit substrate, and are connected to a circuit bus that communicates with the duty cycle and current regulator 12, and the power supply 8. This device is preferred to have an irradiance level of at least 80 μW/cm²/nm. The lens type LED's provide higher intensity levels than LEDs without lenses.

The power supply may be a standard portable power supply capable of generating a relatively low DC voltage, such as 9V, 5V or 3.3V provided by a battery, including a rechargeable battery. Low voltage is desirable for reducing the risk of electrocution for the neonate under treatment. Additionally, the preferred embodiment will also have an AC power adapter enabling longer treatment times without interruptions due to the replacement of batteries used in the DC power source. Appropriate circuitry is provided to control the current to the LEDs.

The current supplied to LEDs is not constant in one embodiment, but is rather pulsed, with a duty cycle of 5-95%. The potted circuitry contains appropriate electronic circuitry to control the timing and activation of LEDs. For example, a circuit 12 providing a direct current duty cycle and current regulator may be provided for intermittent operation. Operating LEDs at such a low duty cycle allows them to be overdriven, i.e., operated with a higher applied current than would be feasible with a constant current. The LEDs are optionally not overdriven because the output of the lens type LED can be brighter than that of the surface mount type used by the current artwork. This also alleviates the need for a heat sink to pull heat away from the device. The allowable ranges for duty cycle, forward current, and pulse width are determined by the operating characteristics of the particular LEDs that are used, according to information provided by the LED manufacturer. Another embodiment of the device would enable the power supply to have external controls that allow a person supervising the phototherapy to control the light intensity, frequency (i.e., pulse width), duty cycle, and color balance if multicolored LED arrays are used. In another embodiment, it may instead have preprogrammed control circuitry that regulates the exposure time and other variables of the treatment. This control circuitry can be programmed using the programming port 73, which is left unpotted during manufacture.

The intermittent power may be supplied with any pulse width satisfying the LED's operating constraints. Intermittent operation at higher power provides significantly more efficient bilirubin photoconversion than does constant operation at lower power. Bilirubin is produced as a by-product of the break down of hemoglobin, which is at high levels after birth. After birth, excess hemoglobin is broken down in a short period of time thereby causing high bilirubin levels (hyperbilirubinemia). Bilirubin enters the skin in significant amounts when the bilirubin concentration is quite high, leading to the characteristic yellowish skin tone. Phototherapy is believed to affect bilirubin in the skin only. When the light is off, the bilirubin concentration in the skin gradually builds up, and it is then converted when the light is on again. Therefore, consistent phototherapy with high irradiance levels is necessary to reduce the overall bilirubin concentrations in the neonate.

The present invention allows the neonate to be fully covered during phototherapy via a gown or blanket, and therefore, the neonate is much more comfortable and less susceptible to hypothermia. The neonate may be wrapped in a blanket, with the present invention inside the blanket against the child's back. The device may also be placed inside clothing. The flexible casing allows deformation of the device as the child is held within a blanket, without damaging or adversely affecting the performance of the device.

In one embodiment, a power supply 8 includes a battery, which may be replaceable or rechargeable, that supplies power to LEDs. The battery or batteries may be held in a pocket or pack that is attached to the outside of the gel material or attached to the care givers belt or clothing. Appropriate circuitry for pulsing the LEDs will be at least partially encapsulated/potted in the gel material. The use of a battery makes the panel fully portable, allowing it to be used when the neonate is transported to and from, and within, the hospital, for example.

Treatment time using the phototherapy device of the present invention varies widely depending upon the conditions of the particular neonate, particularly the serum bilirubin level. However, treatment times are less than with prior methods, which provide much lower irradiance and less surface exposure. The LEDs may be arranged in various configurations. LEDs emitting wavelengths of light other than blue wavelengths may be used, and multiple different wavelengths may be used on the same panel. Phototherapy has been shown to cause a decrease in the endogenous levels of riboflavin, a natural photosensitizer. When irradiated by broad wavelength white or blue light, riboflavin produces singlet oxygen that may react with bilirubin and other organic compounds, causing photodamage and decreased riboflavin levels in developing neonates. Narrow wavelength blue to green LED light is less likely to be absorbed by endogenous photosensitizers and appears to produce fewer side effects, and thus LEDs with higher wavelengths (blue to green) may be included in the device of the present invention.

The device is preferred to be flexible in both the longitudinal and transverse direction. In this embodiment, a substantially transparent flexible material or gel, such as silicone, is poured over the phototherapy panel to encase the LEDs, the printed circuitry, the reflective sheet, and at least part of the control circuitry. In particular, the LEDs are covered with the casing formed by the plastic or gel leaving no spaces, pockets, or channels in the panel. The casing serves as structural support to counter the weight of the patient, thereby preventing the LEDs from being damaged or moved out of proper alignment for maximum efficacy, as well as insuring patient safety and protection from the electrical circuitry. The encasing material is flexible, and allows the device to be deformed so as to follow contours of the infant's body, particularly as the infant is held.

FIG. 9 illustrates an alternative embodiment of the invention. Phototherapy device 30 includes an LED array 32 potted within a polymeric mat 34. Preferably, the polymeric material is silicone, however, the invention is not limited to silicone resins and other transparent flexible resins could be used. The LED array 32 includes a flexible circuit board 36 having an upper side provided with a light reflective surface and a lower side providing a printed circuit board layout for powering a pair of arrays of LEDs 30 and 40. In the embodiment illustrated, half of the LEDs are part of array 38 are of one color and the other half of the LEDs are of a different color. More than two LED color arrays could be used if desired. Printed flexible circuit board 36 has a series of through holes to accommodate the leads of the LEDs, however, the invention is not limited to printed circuit boards having through holes and lens type LEDs. LED arrays can alternatively be made using surface mount LEDs. In this embodiment, the phototherapy device with at least two LED arrays further comprises a multilayer film casing for all of the device components, applied using one of the methods described above.

Power supply cable 42 connects the printed circuit board 36 and the associated arrays of LEDs 30 and 40 to a power supply 44. Preferably, power supply 44 includes a retractable AC connector 46 which is connected to a control board 48 having a rectified circuit to generate DC power. Preferably, the power supply further includes a rechargeable DC battery 50 coupled to a charger circuit on the control board 48. The power supply further includes an on/off switch 52 and a power output level and a color output display. An input device enabling the power and color balance to be varied can alternatively provided on the exterior of the power supply 44 or preset using a non-user accessible input/output set by the medical supply device distributor pursuant to the physician's instruction. The power to the LED arrays 30 and 40 can be independently set using a pair of pulse width modulation circuits to provide the total power output and average color desired. Alternatively a single array of multicolor LEDs could be used to enable the color balance to be varied.

In use, when the baby is at home, the power supply 44 may be plugged into a AC wall outlet. When it is desired to move the baby away from the wall outlet, the power supply can be simply unplugged and the device operated in the DC mode drawing power from batteries 50, which may be rechargeable or non-rechargeable. Preferably, the power supply 44 will be small and compact, comparable to or smaller than the size of a deck of playing cards, so that it can be easily placed in a pocket of a caregiver holding the baby. Of course, a larger power supply could accommodate a large battery and longer use time in the DC mode of operation.

Preferably, control board 48 further includes a timer circuit so that the device can be rendered inoperative after some predetermined number of hours of use or after a predetermined period of time. For example, when a device is provided by a medical equipment supply company, the timer may be set for a period of time sufficient to treat a single infant, for example, 200 hours of use time, two weeks or both. After the time limit has been achieved, the device could be rendered inoperative. This will ensure that the device is in fact only used to treat the single patient for which the device is prescribed. Alternatively, or in combination with a single use time limit, a total elapsed time limit may be established so the device can be taken out of service once it has achieved its useful life, for example 10,000 hours or the like. In this way, the risk of an end of useful life failure of the LED or the power supply of a phototherapy device while in a rental program can be greatly reduced. Preferably, the total elapsed time information can be read and the individual patient time limit can be reset by the medical equipment distributor prior to being provided to a new patient. The LED total life timer continues to accumulate hours in each successive use and is preferably non-resettable.

In another embodiment, when the phototherapy device is to be disposable or used for short time periods, as described above, the predetermined treatment times, individual treatment timer, and total treatment timer can be programmed using a programming port 73, which is not potted during manufacture.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A phototherapy device for treating neonatal hyperbilirubinemia, the device comprising:
flexible circuitry, the circuitry including a plurality of light emitting diodes;
a panel formed of a flexible material surrounding and encasing the plurality of light emitting diodes and at least a portion of the flexible circuitry; and
a multilayer film having at least two layers, the film encasing the flexible circuitry, and the panel, the film being able to withstand heat sterilization at temperatures of at least 121° C. and gamma radiation sterilization.

2. A phototherapy device for treating neonatal hyperbilirubinemia as described in claim 1 further comprising a power supply.

3. A phototherapy device for treating neonatal hyperbilirubinemia as described in claim 1, wherein each of the plurality of light emitting diodes are surrounded by a sheet of reflective material that is encased in the flexible material, and wherein the reflective material is below a top of each of the plurality of light emitting diodes.

4. A phototherapy device for treating neonatal hyperbilirubinemia as described in claim 1, wherein each of the plurality of light emitting diodes are mounted on a flexible substrate that has a substantially white reflective surface, and is fully potted.

5. A phototherapy device for treating neonatal hyperbilirubinemia as described in claim 1, wherein each of the plurality of light emitting diodes are surrounded by a sheet of reflective material that is encased in the flexible material, and wherein the reflective material is below a top of each of the plurality of light emitting diodes, and wherein the reflective material is positioned over the flexible circuitry.

6. A phototherapy device for treating neonatal hyperbilirubinemia as described in claim 1, wherein the plurality of light emitting diodes are lens type light emitting diodes.

7. A phototherapy device for treating neonatal hyperbilirubinemia as described in claim 1, wherein the plurality of light emitting diodes are surface mount light emitting diodes.

8. A phototherapy device for treating neonatal hyperbilirubinemia as described in claim 1, wherein the plurality of light emitting diodes are through hole mount light emitting diodes.

9. A phototherapy device for treating neonatal hyperbilirubinemia as described in claim 1, wherein the plurality of light emitting diodes emit light of a wavelength between 420 and 500 nm.

10. A phototherapy device for treating neonatal hyperbilirubinemia as described in claim 1, wherein the plurality of light emitting diodes emit light of a wavelength between 440 and 480 nm.

11. A phototherapy device for treating neonatal hyperbilirubinemia as described in claim 1, wherein the flexible material is substantially transparent to an efficacious wave length of light emitted by the plurality of light emitting diodes.

12. A phototherapy device for treating neonatal hyperbilirubinemia as described in claim 1, wherein the plurality of light emitting diodes is controlled by a circuit that operates the plurality of light emitting diodes on an intermittent basis.

13. A phototherapy device for treating neonatal hyperbilirubinemia as described in claim 1, wherein the device is programmable to operate for a pre-determined amount of time and then stop emitting light.

14. A phototherapy device for treating neonatal hyperbilirubinemia as described in claim 1, wherein the flexible material comprises silicone gel.

15. A phototherapy device for treating neonatal hyperbilirubinemia as described in claim 14, wherein the flexible circuitry and the circuit that operates the plurality of light emitting diodes on an intermittent basis are at least partially surrounded and encased by the flexible material.

16. A phototherapy device for treating neonatal hyperbilirubinemia, the device comprising:
flexible circuitry, the circuitry including a plurality of light emitting diodes;
a panel formed of a flexible material surrounding and encasing the plurality of light emitting diodes and at least a portion of the flexible circuitry; and
a multilayer film having at least two layers, the film encasing the flexible circuitry, and the panel, a first layer of the multilayer film made of polypropylene and a second layer made of an ethylene/α-olefin interpolymer, the film being able to withstand heat sterilization at 121° C. and gamma radiation sterilization.

17. A method of making a phototherapy device for treating neonatal hyperbilirubinemia, the method comprising:
providing flexible circuitry, the circuitry including a plurality of light emitting diodes;
inserting the flexible circuitry into a bag, the bag comprising a multilayer film having at least two layers, the bag being capable of withstanding heat sterilization at 121° C. and gamma radiation sterilization;
filling the bag with a liquid potting material, the liquid potting material being flexible in its solid state;
evacuating the air from the bag;
sealing the bag; and
curing the liquid potting material.

18. The method of claim 17, wherein the multilayer film comprises a first layer made of polypropylene and a second layer made of an ethylene/α-olefin interpolymer.

19. The method of claim 17 further comprising providing a power supply for the phototherapy device.

20. The method of claim 19 wherein the power supply further includes a power cord that extends from inside the bag to outside the bag, the power cord causing a small gap to be formed when sealing the bag.

21. The method of claim 20 further comprising providing a sealant to the small gap to substantially eliminate it.

22. A method of making a phototherapy device for treating neonatal hyperbilirubinemia, the method comprising:
providing flexible circuitry including a plurality of light emitting diodes;
potting at least a portion of the flexible circuitry in a potting material, the flexible circuitry and the potting material forming a phototherapy panel;
inserting the phototherapy panel into an open-sided IV bag including at least one port, the open-sided IV bag made from a multilayer film, the film being capable of withstanding heat sterilization at 121° C. and gamma radiation sterilization;
sealing the at least one port in the open-sided IV bag; and
sealing the open end of the open-sided IV bag.

23. The method of claim 22 further comprising introducing additional potting material into the bag after the at least one port has been sealed.

24. The method of claim 22 further comprising heat shrinking the phototherapy panel in a film before inserting the phototherapy panel into the open-sided IV bag.
